(19) United States
(12) Patent Application Publication
(10) Pub. No.: US 2022/0105249 A1
(43) Pub. Date: Apr. 7, 2022

(54) X-RAY AND MRI VISIBLE SHAPE MEMORY POLYMER BIOPSY SEALING DEVICE

(71) Applicants: The Texas A&M University System, College Station, TX (US); Shape Memory Medical, Inc., Santa Clara, CA (US)

(72) Inventors: Grace Fletcher, Houston, TX (US); Duncan Maitland, College Station, TX (US); Landon Nash, Amarillo, TX (US); Todd Landsman, Ojai, CA (US); Chung Yeh, San Mateo, CA (US); Edward Ruppel, Saratoga, CA (US); Meghan Reu, Lafayette, CA (US)

(21) Appl. No.: 17/495,009

(22) Filed: Oct. 6, 2021

Related U.S. Application Data
(60) Provisional application No. 63/088,283, filed on Oct. 6, 2020.

(51) Int. Cl. A61L 31/14; A61L 31/18; A61L 31/06; A61K 49/04; A61K 49/18

(52) U.S. Cl. CPC A61L 31/146; A61L 31/18; A61L 2400/16; A61K 49/0438; A61K 49/1896; A61L 31/06

(57) ABSTRACT

An embodiment includes a shape memory polymer (SMP) foam with visibility under both X-ray and magnetic resonance imaging (MRI) modalities. Dual modality visibility is achieved by chemically incorporating monomers with X-ray visible iodine-motifs and MRI visible monomers with gadolinium content. This material platform has the potential to be used in a variety of medical devices.

### CROSS-REFERENCE TO RELATED APPLICATION

This application claims priority to U.S. Provisional Patent Application No. 63/088,283 filed on Oct. 6, 2020 and entitled “X-Ray and MRI Visible Shape Memory Polymer Biopsy Sealing Device”, the content of which is hereby incorporated by reference.

### TECHNICAL FIELD

Embodiments of the invention are in the field of medical devices.

### BACKGROUND

Shape memory polymers (SMPs) are a class of polymeric materials with an ability to change geometry in response to external stimuli. Thermoresponsive SMP materials actuate across a characteristic transition temperature (Tₜᵣₐₙₛ) that is based on polymeric structure. Temperature elevation above the polymer's Tₜᵣₐₙₛ enables deformation into a secondary geometry. Maintaining the deformation while cooling below Tₜᵣₐₙₛ temporarily programs this secondary shape. The unconstrained material will return to the primary geometry when heated back above the Tₜᵣₐₙₛ. This behavior enables a variety of biomedical applications such as conformal bone defect grafts, self-tightening sutures, and devices for minimally-invasive procedures.

Porous polymeric scaffolds are useful in a variety of applications, particularly those requiring tissue ingrowth, as the porous network and large surface area promote cellular infiltration, attachment, and rapid clot formation. A class of biocompatible thermoset polyurethane SMPs using aliphatic isocyanates that can be gas-blown into low density, porous morphologies was originally envisioned for use in biomedical applications. To utilize these properties, these foams and modifications thereof have been implemented in a variety of embolic device designs. In this case, the shape memory behavior coupled with the porous foam morphology enables minimally-invasive delivery of medical devices as the foams can be compressed to low diameters for catheter-guided delivery.

### BRIEF DESCRIPTION OF THE DRAWINGS

Features and advantages of embodiments of the present invention will become apparent from the appended claims, the following detailed description of one or more example embodiments, and the corresponding figures. Where considered appropriate, reference labels have been repeated among the figures to indicate corresponding or analogous elements.

FIG. 1 addresses hydroxyl or carboxylic acid containing monomers and their role in synthesis of a porous SMP foam with dual contrast on X-ray and MRI modalities in an embodiment.

FIG. 2A shows SEM Images of selected foam compositions highlighting pore size and morphology (scale bar 2 mm for all images) in an embodiment. FIGS. 2B and 2C show zoomed in SEM images highlighting unique morphological features in embodiments. The circles in FIG. 2B denote the thinning of pore membrane due to the addition of cell opener in the 20 AT 0 GPA composition. The arrows in FIG. 2C denote thick struts in composition 20 AT 0.01 GPA.

FIG. 3 includes ATR-FTIR spectroscopy results for a selection of compositions with peaks of interest identified (C═O Urethane 1685 cm-1, C═O Urea 1650 cm-1, Amide II 1510 cm-1) in an embodiment.

FIG. 4A discloses unconstrained expansions of 6 mm diameter foam punches upon exposure to a 37° C. water bath in an embodiment. Images were analyzed every minute for the first 10 minutes and at 5 minute intervals until 30 minutes, then at 15 minute intervals for the remaining time points. FIG. 4B discloses images from the water bath at the 15, 30, and 45 minute time points to show expansion of foams over time in an embodiment.

FIGS. 5A-C discloses MR images using T₁-weighted parameters (TE=30 msec, TR=500 msec) in embodiments. FIGS. 5A and 5B show coronal MR images of compositions as labelled with oil and water controls. FIG. 5C shows transverse MR images of compositions as labelled.

FIG. 6 (see portion (a)) shows selected SMP foams with 20 mol % ATIPA and varying amounts of GPA imaged on OrthoScan C-arm in an embodiment. FIG. 6 (see portion (b)) shows an image that was performed through a ½″ Al plate to simulate imaging through bone in an embodiment. Foam samples in columns are labeled with thickness for cubes and crimp state of 6 mm diameter cylindrical biopsy punch samples.

FIG. 7 shows data for 6 mm cylindrical foam punch embodiments of multiple compositions in expanded and crimped form for each of the X-ray images to determine their relative opacity: (Blue) Baseline X-ray image; (Orange) Attenuated X-ray image taken through ½″ aluminum plate.

FIG. 8 shows cell viability calculated from RFU in resazurin assay performed upon 3T3 cell exposure to media extracts with embodiment compositions of SMP foams. Extractions and cytocompatibility assays (n=6 wells for each assay) were repeated in triplicate for a total sample size of 18. Red line indicates 1030 or threshold where 70% of cells are alive. All compositions were above the 1030 threshold.

FIG. 9 shows a biopsy system in an embodiment.

FIG. 10 addresses chemical bonding to provide an X-ray and MRI visible foam in an embodiment.

### DETAILED DESCRIPTION

Reference will now be made to the drawings wherein like structures may be provided with like suffix reference designations. In order to show the structures of various embodiments more clearly, the drawings included herein are diagrammatic representations of structures. Thus, the actual appearance of the fabricated structures, for example in a photo, may appear different while still incorporating the claimed structures of the illustrated embodiments (e.g., walls may not be exactly orthogonal to one another in actual fabricated devices). Moreover, the drawings may only show the structures useful to understand the illustrated embodiments. Additional structures known in the art may not have been included to maintain the clarity of the drawings. For example, not every layer of a device is necessarily shown. “An embodiment”, “various embodiments” and the like indicate embodiment(s) so described may include particular features, structures, or characteristics, but not every embodiment necessarily includes the particular features, structures, or characteristics. Some embodiments may have some, all, or none of the features described for other embodiments. “First”, “second”, “third” and the like describe a common object and indicate different instances of like objects are being referred to. Such adjectives do not imply objects so described must be in a given sequence, either temporally, spatially, in ranking, or in any other manner. “Connected” may indicate elements are in direct physical or electrical contact with each other and “coupled” may indicate elements co-operate or interact with each other, but they may or may not be in direct physical or electrical contact. Phrases such as “comprising at least one of A or B” include situations with A, B, or A and B.

Regarding the above work on porous polymeric scaffolds, Applicant determined a major limitation of the SMP materials is that they are not visible on medical imaging modalities.

This is important because many medical procedures are guided by or monitored using X-ray fluoroscopy. Since polymers lack the high density required for radiopacity, there has been extensive work to achieve X-ray visibility through a variety of chemical and physical additive approaches. Use of nanoparticle and microparticle fillers to improve opacity in the SMP foams has been explored, but Applicant determined these composite methods can alter bulk thermal and mechanical properties. Another possible approach is chemically incorporating a radiopaque monomer. Recently, Lex et al. reported on polyesters with enhanced X-ray contrast derived from custom iodinated monomers. Still, such work did not concern, for example, maintaining or managing polyurethane foam mechanics/characteristics (e.g., expansion, tensile strength, and X-ray visibility) while still making such foams radiopaque.

Applicant previously achieved adequate visualization of thermoset SMP foams by chemically incorporating iodine-containing motifs. The radiopaque monomer used in various embodiments is 5-amino-2,4,6-triiodoisphthalic acid (ATIPA) which contains a triiodobenzene ring with an amine group and two carboxylic acid groups for incorporation into the polyurethane network. Triiodobenzene iodine motifs are commonly used in biomedical contrast agents such as Iohexol and Iopamidol due to their absorption of X-rays. While X-ray is a common medical imaging modality, Applicant determined it does necessitate patient exposure to ionizing radiation which can be a concern for pediatric populations and patients requiring imaging often. Furthermore, Applicant determined it does not provide physicians with the same level of dynamic anatomical information as other imaging modalities.

Magnetic resonance imaging (MRI) is a medical imaging modality with the benefits of no ionizing radiation exposure and improved soft tissue contrast. A strategy for imparting MRI visibility into materials while avoiding device heating during imaging is to generate positive contrast with a passive technique. The most common manifestation of this approach involves MR contrast agents, most often gadolinium-based contrast agents (GBCAs), incorporated into the device in some manner to utilize the T₁-shortening effects of these agents. Younis et al. grafted a GBCA onto a poly(methyl methacrylate) based copolymer which was ultimately utilized as a coating for a polypropylene mesh. Other approaches for MR visibility in medical devices include incorporating other paramagnetic components; for example, Brocker et al. investigated the use of iron oxide woven into a polypropylene mesh material to achieve MRI visibility in devices.

In general, these MRI contrast agents rely on paramagnetic effects to shorten T₁ relaxation times, decreasing T₁ saturation effects and leading to increased signal intensity when imaging using T₁-weighted sequences. Gadolinium is commonly used in this way to generate MRI contrast because it enhances the proton relaxation of surrounding water and its paramagnetism is preserved when complexed with or conjugated to other molecules. Weems et al. previously incorporated the monomer diethylenetriaminepentaacetic (DTPA) acid gadolinium (III) dihydrogen salt hydrate also known as gadopentetic acid (GPA) into thermoset SMP foams based on trimethyl hexamethylene diisocyanate (TMHDI). The carboxylic acid groups on this monomer allow for incorporation into the polymer backbone. This approach also utilized the same structure of gadolinium chelate in the commercially available GBCA Magnevist, which provides a reference for acceptable, nontoxic levels of gadolinium.

Embodiments herein address SMP materials with both X-ray and MR visibility that can be modified for multiple applications. A few groups have previously successfully modified polymers containing dual-modality contrast including Goodfriend et al. who synthesized a bioresorbable polyester named poly(gadodiamide fumaric acid) that is X-ray visible in its liquid coating form and MRI-visible in nanoparticle form. Weems et al. also physically incorporated iron oxide nanoparticles for enhanced SMP visibility on both X-ray and MR imaging modalities. Still, such work did not concern, for example, maintaining or managing polyurethane foam mechanics/characteristics (e.g., expansion, tensile strength, and X-ray visibility) while still making such foams radiopaque and MRI visible.

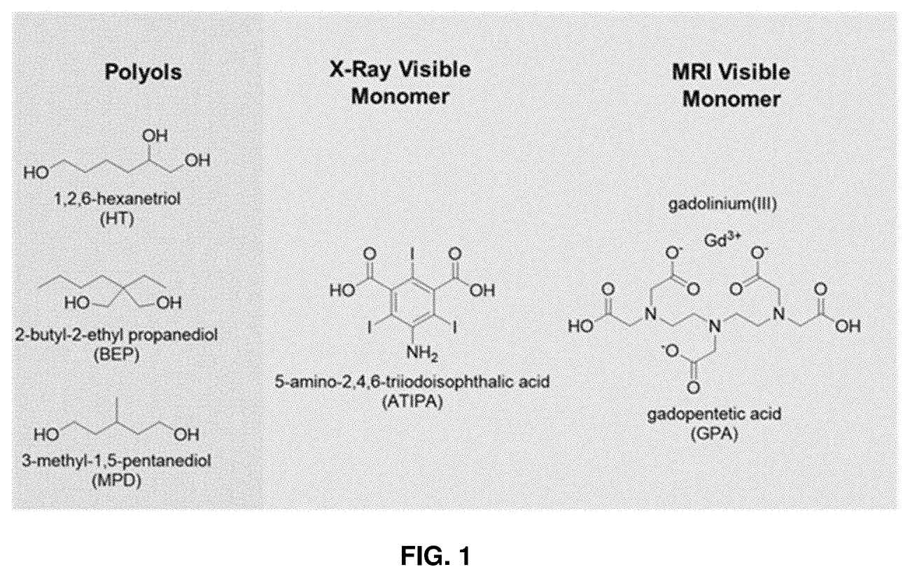

Embodiments provide a material platform that can be used in many applications requiring guided delivery and follow-up monitoring. Combining ATIPA and GPA systems into a single foam creates a new porous shape memory polymer with X-ray and MR visibility imparted by chemical modifications. A material used in embodiments is an amorphous SMP where the T_{g} control is dependent on the isocyanate component and the amount of hexanetriol. The addition of GPA to this material system will increase T_{g} due to increased rigidity. Applicant determined porosity is an important feature of these shape memory polymers and must be balanced with the correct amount of contrast monomers to ensure visibility in both compressed and expanded forms of the foam. FIG. 1 shows the role of each monomer in the material system. Morphological, chemical, and thermomechanical characterizations were performed on multiple compositions. X-ray and MR imaging pilot studies were performed to verify visibility. Furthermore, extractions under simulated use conditions and indirect cytocompatibility studies were conducted to assess toxicity.

2. Results

The SMP foam compositions synthesized for the studies were named according to the convention delineated in Table 1. The compositional names arise from the amounts of ATIPA (X-ray visible monomer) and GPA (MR visible monomer) in each composition. These are reported as mol % of functionalities reactive with isocyanates (OH, COOH, NH2). The isocyanate used in embodiments is hexamethylene diisocyanate (HDI) but other isocyanates are applicable in other embodiments.

TABLE 1
Hydroxyl components of shape memory polymer compositions
synthesized for use in all studies.

| | ATIPA | GPA | MPD | BEP | HT |
|---|---|---|---|---|---|
| Composition | (eq %) | (eq %) | (eq %) | (eq %) | (eq %) |
| 20 AT 0 GPA | 20 | 0 | 40 | 20 | 20 |
| 20 AT 20 GPA | 20 | 20 | 20 | 20 | 20 |
| 20 AT 10 GPA | 20 | 10 | 23.3 | 23.3 | 23.3 |
| 20 AT 5 GPA | 20 | 5 | 25 | 25 | 25 |
| 20 AT 2.5 GPA | 20 | 2.5 | 25.8 | 25.8 | 25.8 |
| 20 AT 1 GPA | 20 | 1 | 26.3 | 26.3 | 26.3 |
| 20 AT 0.05 GPA | 20 | 0.05 | 26.7 | 26.7 | 26.7 |
| 20 AT 0.025 GPA | 20 | 0.025 | 26.7 | 26.7 | 26.7 |
| 20 AT 0.01 GPA | 20 | 0.01 | 26.7 | 26.7 | 26.7 |
| 20 AT 0.001 GPA | 20 | 0.001 | 26.7 | 26.7 | 26.7 |

2.1 Physical Characterization

Physical characterization included density and pore size measurements. Table 2 contains the SMP foam densities and pore sizes for all compositions made. Pores were measured in the axial and transverse directions.

TABLE 2
Physical and thermomechanical properties of SMP foams.
Measurements are reported as mean ± standard deviation for the indicated sample
size in top row.

| | Density | Pore Sizes | Dry T_{g} | Wet T_{g} | Gel Fraction |
|---|---|---|---|---|---|
| | (g/cm³) | (μm) | (° C.) | (° C.) | (%) |
| Composition | n = 3 | n = 30 | n = 3 | n = 3 | n = 3 |
| 20 AT 0 GPA | 0.076 ± 0.007 | Axial 1447 ± 344 | 47.5 ± 0.9 | 32.1 ± 1.6 | 97.2 ± 0.3 |
| | | Trans 1523 ± 329 | | | |
| 20 AT 20 GPA | 0.123 ± 0.007 | Axial 702 ± 108 | 66.8 ± 1.0 | 39.2 ± 1.1 | 98.8 ± 0.3 |
| | | Trans 711 ± 118 | | | |
| 20 AT 10 GPA | 0.039 ± 0.004 | Axial 1853 ± 505 | 66.9 ± 0.3 | 37.1 ± 0.9 | 97.5 ± 0.1 |
| | | Trans 1493 ± 311 | | | |
| 20 AT 1 GPA | 0.093 ± 0.003 | Axial 920 ± 199 | 64.0 ± 0.9 | 36.0 ± 1.0 | 98.0 ± 0.1 |
| | | Trans 744 ± 122 | | | |
| 20 AT 0.01 GPA | 0.128 ± 0.004 | Axial 1408 ± 93 | 59.8 ± 0.5 | 36.4 ± 0.7 | 98.6 ± 0.3 |
| | | Trans 798 ± 69 | | | |
| 20 AT 0.001 GPA | 0.114 ± 0.010 | Axial 1211 ± 364 | 54.9 ± 1.1 | 37.8 ± 1.8 | 98.3 ± 0.4 |
| | | Trans 1051 ± 229 | | | |

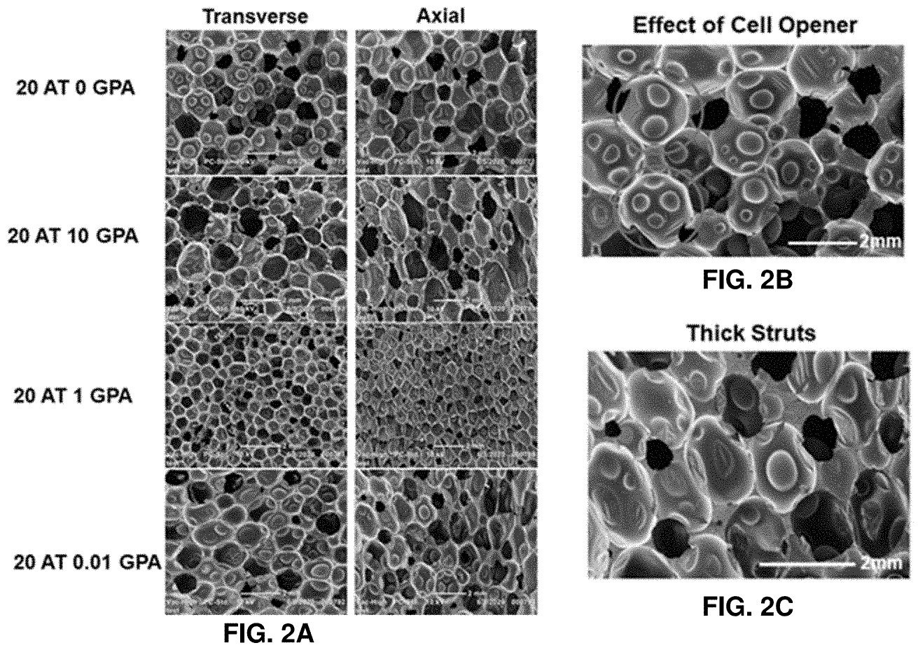

The SEM images of the foam (FIGS. 2A, 2B, 2C) show the effect of the cell opener as well as the thick struts in the ATIPA GPA foams. Pore size and morphology varied in compositions due to pre-polymer viscosity and amount of GPA. Foams with larger pores have elongated pores in the axial (foaming) direction. Thick struts were visible in both the 20 AT 0.01 GPA and 20 AT 0.001 GPA compositions.

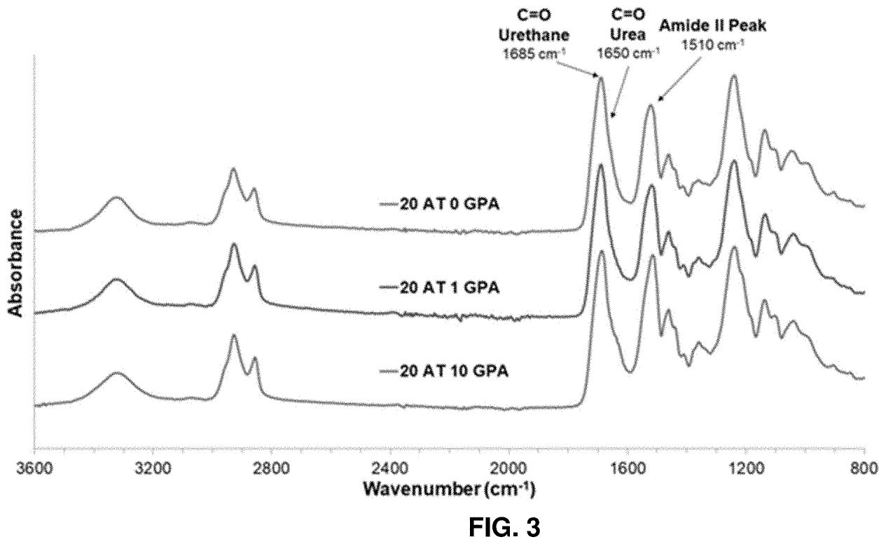

FIG. 3 shows the ATR-FTIR spectroscopy for selected compositions. A strong urethane C═O peak is present at 1685 cm-1 for all compositions, so spectra were normalized to this peak. Compositions exhibit a urea C═O shoulder at 1650 cm-1 from the primary amine on ATIPA's reaction with isocyanate. The 20 AT 10 GPA composition has the strongest amide II peak at 1510 cm-1 due to higher carboxylic acid content in the synthesis from the GPA monomer.

2.2 Thermomechanical Characterization

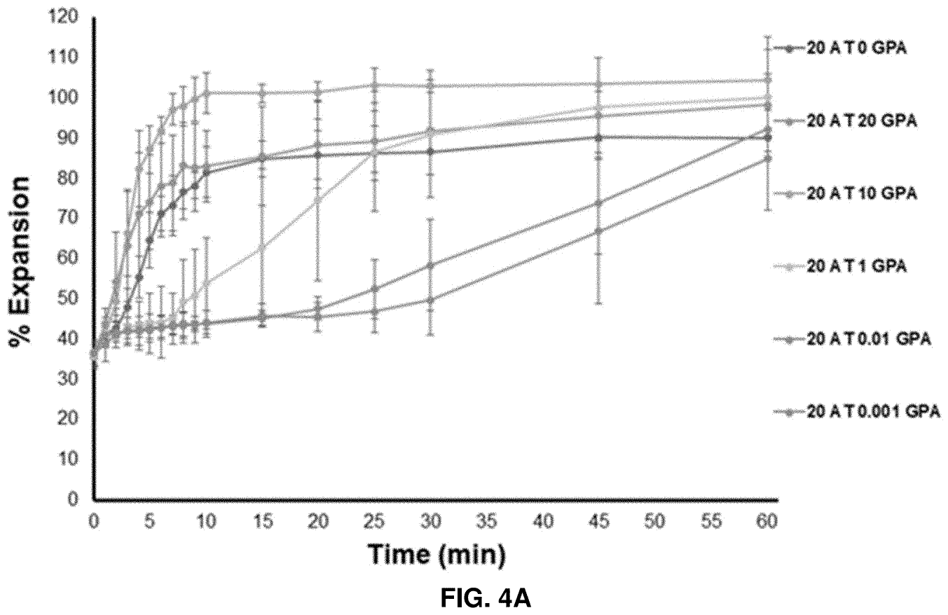

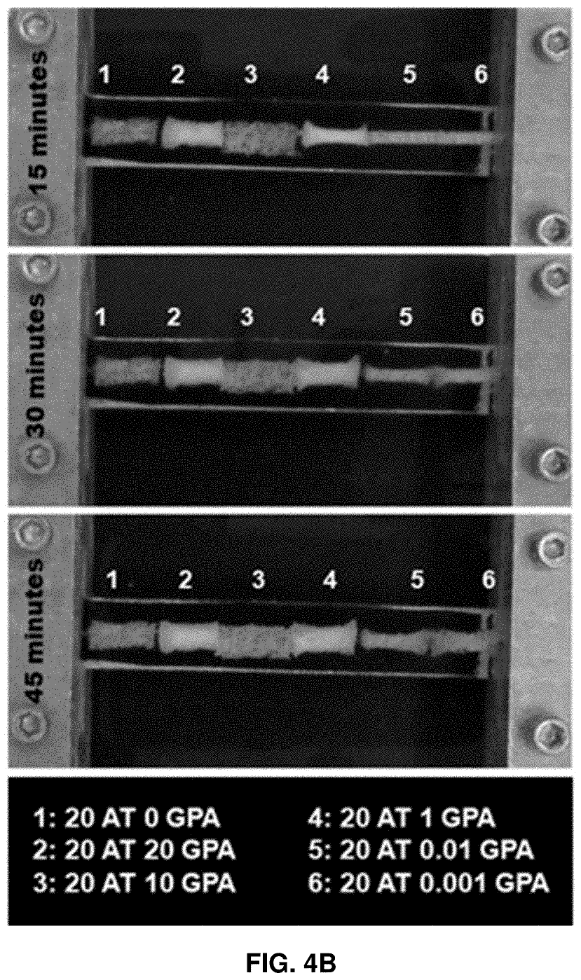

Glass transition temperatures (Table 2) increased with increasing GPA content. However, all compositions also demonstrated water plasticized transition temperatures (wet T_{g}'s) close to body temperature. FIGS. 4A and 4B summarize the unconstrained expansion behavior of the foams in a 37° C. water bath. The 20 AT 10 GPA foam had the most rapid expansion, reaching 100% expansion at ˜10 minutes. The 20 AT 0 GPA foam did not reach 100% expansion but also reached its terminal diameter quickly (˜15 minutes). The 20 AT 0.01 GPA and 20 AT 0.001 GPA foams expanded the slowest and did not reach 100% expansion at 60 minutes. The expansion behavior of the foams seems to be related to both foam density and GPA content increasing hydrophilicity of the foam.

Tensile tests were performed on rectangular foam samples (n=3) affixed to wooden blocks. The ultimate tensile strength (UTS) was calculated from the peak stress on a stress-strain curve (Table 3). UTS and stiffness increases correlate to increasing SMP foam density, except in the case of the 20 AT 20 GPA formulation. This is likely due to increased loading of GPA.

TABLE 3
Ultimate tensile strength (UTS) for compositions with varied GPA
content.

| | Ultimate Tensile Strength |
|---|---|
| Composition | (kPa) |
| 20 AT 0 GPA | 356.5 ± 36.3 |
| 20 AT 20 GPA | 455.2 ± 92.2 |
| 20 AT 10 GPA | 203.7 ± 41.1 |
| 20 AT 1 GPA | 751.8 ± 126.8 |
| 20 AT 0.01 GPA | 927.4 ± 101.0 |
| 20 AT 0.001 GPA | 1061.1 ± 71.8 |

2.6 Magnetic Resonance Imaging

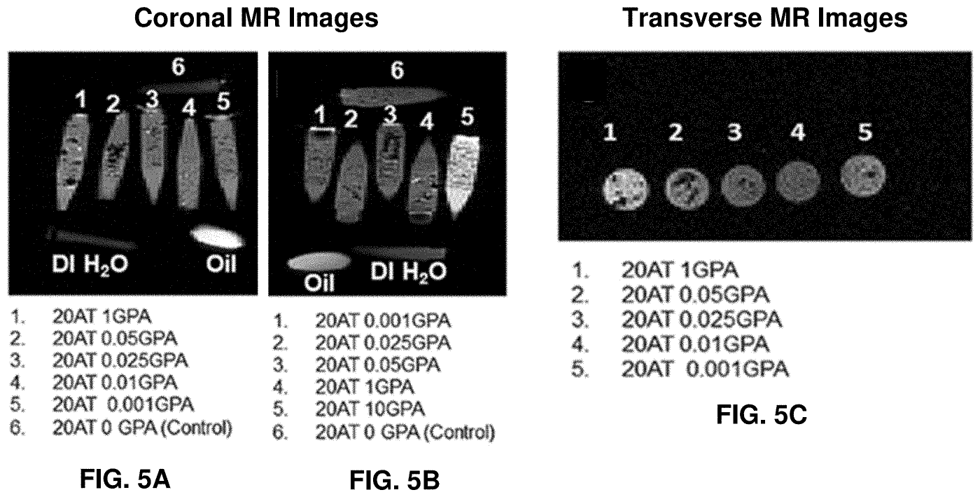

The T₁-weighted MR images were taken in both coronal and transverse planes. Low GPA concentration foams (FIG. 5A) did not enhance foam visibility in the coronal view relative to the positive oil control and negative DI water control. However, the 20 AT 10 GPA foam (#5 on FIG. 5B) showed a marked brightening effect around the foam and the vial in the coronal view. This intensity exceeds that of the fiducial control (oil capsule) and the other foams in the same image. There is a slight brightening effect of the 20 AT 1 GPA (#1 on FIG. 5C) foam in the transverse view. 20 AT 20 GPA composition is excluded from MR images because we observed darkening due to high concentrations of gadolinium.

2.7 X-Ray Imaging

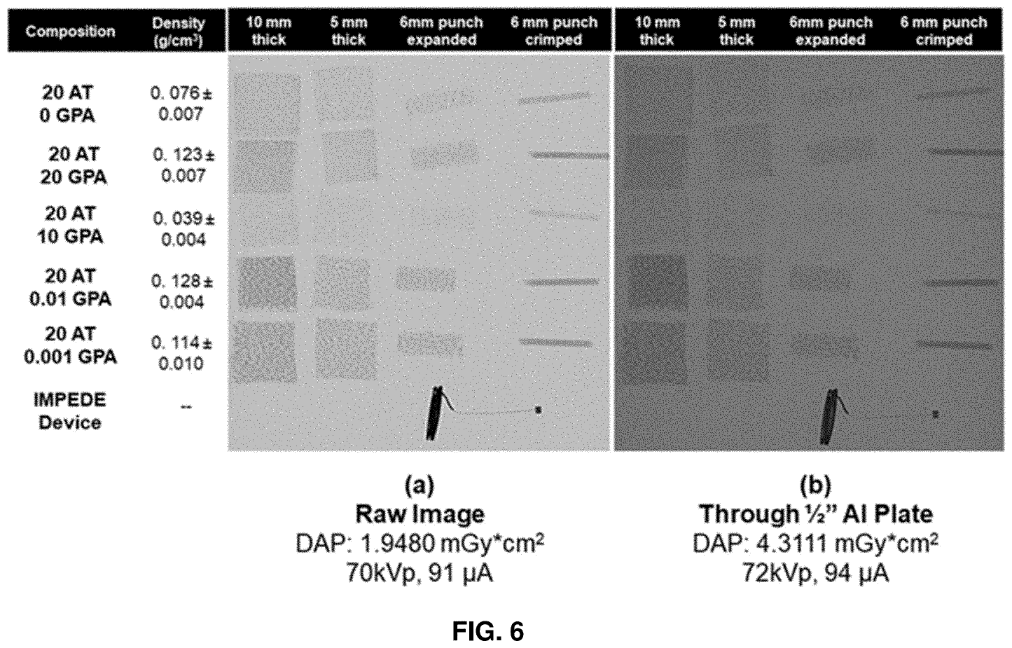

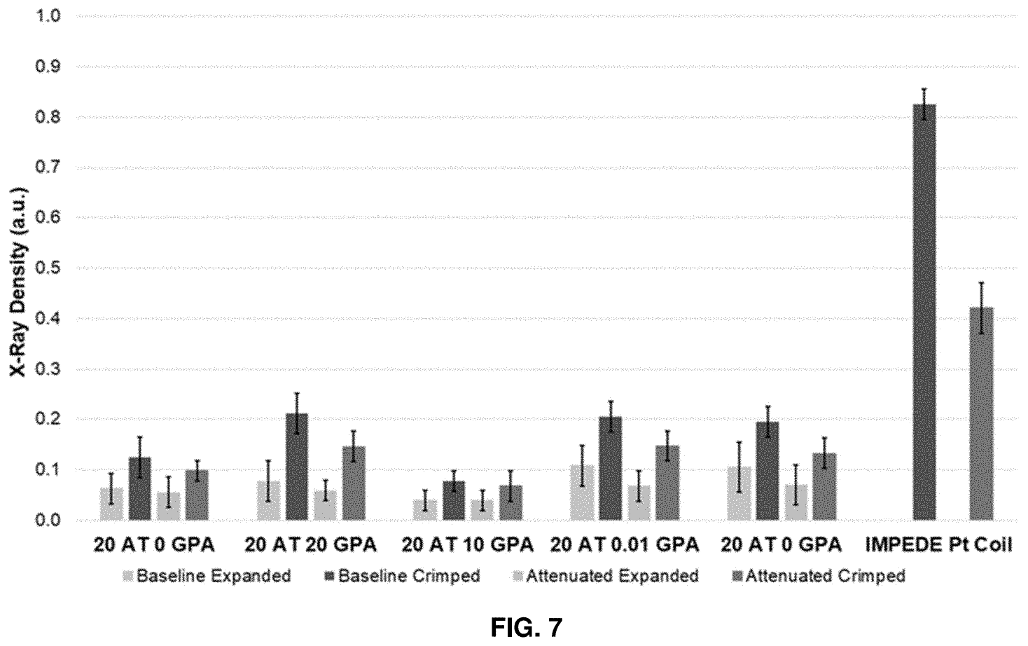

The X-ray images were performed on multiple foams with varying densities. The images were taken both directly and through a ½″ aluminum plate to mimic attenuation from bone (e.g., skull). The X-ray images obtained (FIG. 6) were analyzed by measuring the pixel intensity 60 points on the sample of interest (FIG. 7). This was performed after background removal processing step in ImageJ. The ATIPA-GPA foam X-ray pixel intensity values were compared to those for the platinum coil (Pt Coil) of the IMPEDE device (Shape Memory Medical Inc, Santa Clara, Calif.), which was analyzed as a control for the opacity of metallic device components.

FIG. 7 shows the relative X-ray densities of the 6 mm foam samples in crimped and expanded forms. The images taken with a ½″ aluminum plate on top all have lower X-ray densities than their raw image counterparts, which is expected due to X-ray attenuation. Foam density was the largest factor in X-ray opacity since all compositions contained the same amount of ATIPA monomer (20 mol %). The 20 AT 20 GPA, 20 AT 0.01 GPA, and 20 AT 0.001 GPA compositions are the highest density materials and show the greatest X-ray density in both the baseline and attenuated images. Porous morphology of the 20 AT 0.01 GPA and 20 AT 0.001 GPA foams is visible in the X-ray images due to thicker struts.

2.8 Mass Spectroscopy of Extractables

Extractions were performed in DI water and 50% ethanol extraction vehicles to identify the amount of extractable and leachable gadolinium under simulated use conditions. The highest reported extraction concentration of 7510 ng/mL was reported for the 20AT 20 GPA composition extracted in 50% ethanol, equivalent to a total extracted weight of 75.1 mg for the sample. This is approximately 8 times more gadolinium than the equivalent extraction in DI water.

TABLE 4
ICP-MS results for the ATIPA-GPA foam samples in different
extraction vehicles. Results are
displayed as Gd concentration ± uncertainty.1

| | DI Water | 50% Ethanol |
|---|---|---|
| | Extract Gd | Extract Gd |
| | Concentration | Concentration |
| Composition | (ng/mL) | (ng/mL) |
| 20 AT 20 GPA | 938 ± 47 | 7510 ± 380 |
| 20 AT 10 GPA | 259 ± 13 | 2160 ± 110 |
| 20 AT 1 GPA | 116 ± 6 | 419 ± 21 |
| 20 AT 0.01 GPA | 2.4 ± 0.1 | 38 ± 2 |
| 20 AT 0.001 GPA | <0.1* | 0.30 ± 0.02 |

1Uncertainties provided at the 1 s (67% confidence level)
*Quantitation limit

2.9 Indirect Cytocompatibility

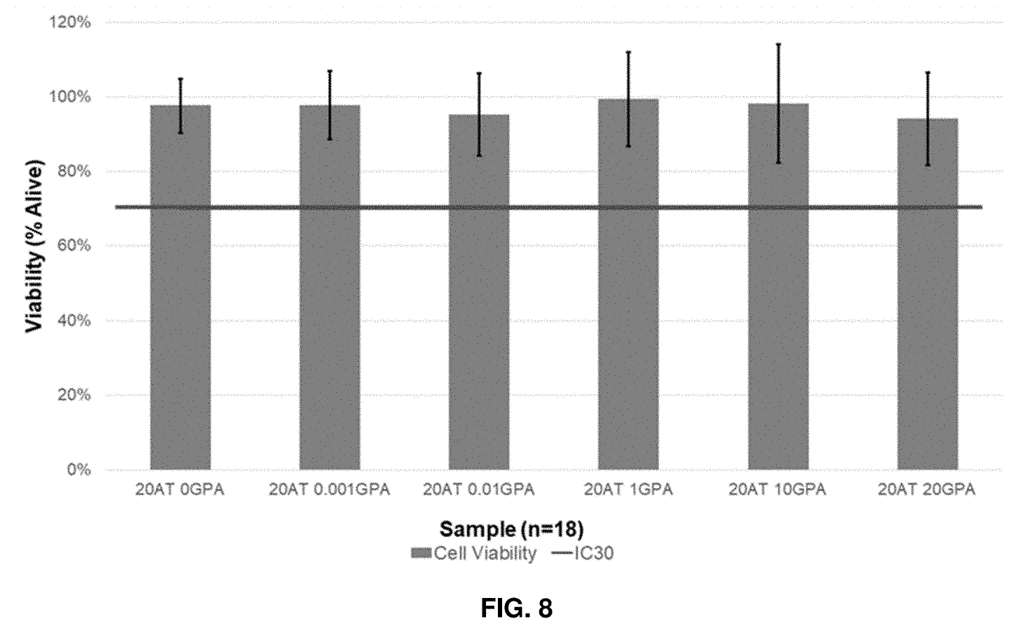

Media extracts were exposed to 3T3 cells in order to determine the compositions' cytocompatibility and results are displayed in FIG. 8. Cell viability was determined using a resazurin assay and calculated from Equation 3. While the composition with the highest GPA content (20 AT 20 GPA) did have the lowest cell viability, all compositions were above the IC30 threshold indicated by the red line on FIG. 8.

3. Discussion

In general, as GPA content decreased, the density of the polymers increased. This is due to the GPA monomer's carboxylic acid groups, which acted as an additional blowing agent due to the generation of carbon dioxide upon reacting with isocyanates. The exception to this trend was the 20 AT 20 GPA composition with a higher density. This level of GPA in the 20 AT 20 GPA composition increased the reactive mixture's viscosity, which decreased the average pore size and increased ultimate material density, despite increased carboxylic acid chemical blowing.

Glass transition temperatures (Table 2) increased with increasing GPA content. This trend was also observed in Weems et al. and is due to restricted chain mobility in the polymeric structure with increasing rigid GPA monomer incorporation. However, all compositions demonstrated water plasticized transition temperatures close to body temperature. The materials' high dry Tg's are beneficial in terms of medical device shipping and storage conditions to maintain secondary geometries without premature actuation.

The unconstrained expansion behavior of the foams in a 37° C. water bath (FIGS. 4A and 4B) was more affected by material density and pore size than composition Tg. The lowest density 20 AT 10 GPA foam was the quickest to expand. This is due to higher density materials contributing a more significant physical diffusion barrier to moisture plasticization. Moisture plasticization depresses the transition temperatures due to disruption of hydrogen bonding. All compositions had moisture plasticized transitions close to body temperature, therefore linking expansion rate to plasticization rate.

The pilot MR imaging studies provided a range of acceptable concentrations of gadolinium. Gadolinium causes shortening of both T₁ and T₂. When using T₁-weighted imaging sequences, the T₁ shortening manifests as the clinically desired image brightening, while T₂ shortening will result in image darkening. Depending on the concentration of gadolinium used, one of these effects will be dominant. The 20 AT 20 GPA composition had too high of a concentration of gadolinium and T₂ effects dominated, so it was excluded from further study. While brightening was seen for the 20 AT 10 GPA and 20 AT 1 GPA compositions, the pilot MR images are somewhat limited since all materials were imaged in vials containing DI water. Although DI water is not entirely representative of T₁ and T₂ tissue MR properties, general trends of increased brightening for GBCA incorporated compositions should still be clinically relevant for T₁-weighted imaging sequences. In general, visibility of any foam is expected to be dependent on the surrounding tissue properties as well as the exact imaging parameters used.

While reaction conditions were kept constant between compositions, the differences in viscosity of monomer solution and amount of the GPA monomer resulted in different material density and pore sizes. Since material density played a large role in material performance (expansion, tensile strength, and X-ray visibility), it will likely be tuned for specific performance outcomes depending on the chosen application. For example, larger pore and lower density foams will expand faster, but have lower mechanical strength and require a higher loading of contrast agents for visibility, whereas higher density foams will expand slower, but have superior mechanical strength and visibility. Ideal material density can be tailored in each composition by changing the amount of physical blowing agent and reactive mixture viscosity.

The extractions of the 20 AT 20 GPA composition using a 50% ethanol extraction vehicle in simulated use conditions had the highest amount of extracted gadolinium, however it was well below the maximum doses administered for adult and pediatric populations. Magnevist is prepared at a concentration of 469 mg/mL and administered at a maximum dose of 0.6 mL/kg for adult patients and 0.4 mL/kg for pediatric patients. Using these dosing guidelines, almost 19 devices with a similar footprint (6 mm diameter, 25 mm length cylinder) as the extracted foam sample could be delivered to a 10 kg pediatric patient. In adult patients of 50 kg, approximately 187 devices could be delivered while being in the dosing safety threshold (CDC currently reports an average weight of women and men in the US of ˜77 kg and 89 kg respectively).

Indirect cytocompatibility study results complement the extraction ICP-MS data. The 20 AT 20 GPA composition had the lowest average cell viability (94.1±12.3%). However, all compositions were above the 1030 threshold. Gadolinium is toxic because a free Gd³⁺ ion can interfere with normal biological processes due to its similarity to the Ca²⁺ ion. Chelated versions of gadolinium are used to prevent this.

The approaches for imparting X-ray and MRI visibility to SMP foams through chemical modification described herein could be translated to many material systems. Additionally, SMP foam properties could be tuned for specific device requirements. These foams could be incorporated into current embolic device designs to reduce or eliminate metallic components used for visualization. The foam itself being visible provides more information about the expansion and volume-filling properties of the devices. An example of a new application amenable to the developed material system is a biopsy sealing device. The benefit of the X-ray and MRI visibility for this application would be that the device could be imaged during delivery and the polymer plug could act as a fiducial marker to designate the tract where the biopsy was taken from. This material system also has potential to be a fiducial marker in other tissue engineering applications requiring MRI or X-ray visible tissue scaffolds. For biodegradable formulations, the diminishing material visibility under different imaging modalities can be an indicator for material degradation and clearance from the body. The material can also be used as an anatomical indicator used in conjunction with radiation therapies or radiomedicine applications.

4. Materials and Methods

4.1 Materials

All chemicals were used as purchased unless otherwise indicated. 3-methyl-1,5-pentanediol, 2-butyl-2-ethyl propanediol, 1,2,6-hexanetriol, 5-amino-2,4,6-triidoisosphthalic acid (95%), and gadopentetic acid (97%) were all purchased from Sigma Aldrich. Hexamethylene diisocyanate was purchased from TCI. The cell opener Tegostab B8523 and surfactant DC1990 were provided by Evonik. Tetrahydrofuran was from EMD Millipore and was stored over molecular sieves. Isopropanol was purchased from VWR. Enovate was purchased from Honeywell.

4.2 SMP Foam Synthesis

All foam compositions used in the study are listed in Table 1. Foams were synthesized using a classic hydroxyl (OH) and isocyanate (NCO) polyurethane reaction scheme. The hydroxyl components used were 3-methyl-1,5-pentanediol (MPD), 2-butyl-2-ethyl propanediol (BEP), and 1,2,6-hexanetriol (HT). The isocyanate component was hexamethylene diisocyanate (HDI). The X-ray visible monomer used was 5-amino-2,4,6-triiodosphthalic acid (ATIPA). The MRI visible monomer was diethylenetriaminepentaacetic acid gadolinium (III) dihydrogen salt hydrate also known as gadopentetic acid (GPA). All monomers were used as received and premixes were prepared in a glovebox.

The foaming procedure was adapted from Nash et al to accommodate addition of gadolinium. The hydroxyl components (0.6 molar eq.) of the OH premixes (MPD, BEP, HT, ATIPA) were measured out into a 150 mL Flacktek mixing cup two days before foaming. They were mixed in a Flacktek high speed mixer (Model DAC 150 FVZ-K, Flacktek Inc, Landrum, S.C.) for 30 seconds at 3540 rpm and placed in an oven at 50° C. After 1 hour, the cup was mixed again for 30 seconds and remained in the 50° C. oven overnight.

Similarly, the hydroxyl components (0.4 molar eq.) of the NCO premixes (as well as ATIPA) were added to a separate Flacktek mixing cup two days prior to foaming. They were mixed in a Flacktek high speed mixer for 30 seconds at 3540 rpm and placed in an oven at 50° C. After 1 hour, the cup was mixed again for 30 seconds and remained in the 50° C. oven overnight. The following day, the contents were mixed another 30 seconds before adding the diisocyanate equivalents (1.02 molar eq.). The mixture was mixed in a Flacktek high speed mixer for 5 minutes at 3540 rpm and placed on a shaker plate at room temperature overnight or until the mixture's viscosity increased to a honey-like consistency.

The surfactant DC1990 (4 wt %) and the cell opener Tegostab B 8523 (0.025 wt %) were added to the NCO premix prior to foaming and mixed for 30 seconds. GPA was added to the OH premix and mixed for 1 minute immediately before foaming. The OH premix was added to the NCO premix and mixed for 30 seconds. A physical blowing agent, Enovate, was added to that mixture and mixed for 15 seconds. The cup was moved to an oven at 90° C. and allowed to cure for 20 minutes. The foam was allowed to cool in a fume hood and the skin was removed with a razor blade before an overnight post-cure in a 50° C. oven.

Foam samples were placed in a jar and submerged in DI water and sonicated for three 30 minute cycles to rinse before adding isopropyl alcohol (IPA) in a 20:1 IPA:foam volume ratio. The jars were subjected to three 30 minute intervals in a sonication bath, switching out IPA between intervals. They were dried overnight at 100° C. in a vacuum oven and stored with desiccant before characterization took place unless otherwise noted.

4.3 Physical Characterization

4.3.1 Density

Cubes (˜1 cm³) from each foam block were used for density measurements. The dimensions of the block were measured (n=3) with calipers and volume was calculated. Mass of the blocks was measured on a balance (n=3). Density was calculated using averaged mass and volumes for the samples.

4.3.2 Pore Morphology

Foam slices were cut in the axial and transverse directions in the middle of the foam, mounted on carbon tape affixed to an imaging stub, dried, and sputter coated. Slices were imaged using a JEOL JCM-5000 Neoscope scanning electron microscope (JEOL USA Inc., Peabody, Mass.). 30 pores were measured per slice direction using ImageJ software.

4.3.3 Fourier Transform Infrared Spectroscopy (FTIR)

Foam samples were cut and compressed to be measured using a Bruker Spectrometer (Bruker, Billerica, Wash.). Spectra were obtained via a germanium attenuated total reflectance (ATR) probe. Thirty-two background scans were performed prior to each sample measurement. Samples were measured using 64 scans and the resulting spectra were corrected for atmospheric compensation using Bruker OPUS software and exported to Excel where they were normalized to the urethane peak.

4.4 Gel Fractions

Gel fraction was performed on cleaned and dried foams to determine the extent of crosslinking. The initial cleaning step removes excess surfactant and catalysts. Original dry weight was measured using a balance (Mettler Toledo, Columbus, Ohio). Foam blocks were incubated in THF for 3 days at 50° C. Foams were dried at 50° C. under vacuum for 2 days and measured to get the final weight. Gel fraction was calculated using the equation:

$$\frac{{m}_{1}}{{m}_{0}} \times 100 = {G}_{f} . \qquad ( 1 )$$

4.5 Thermomechanical Characterization

4.5.1 Differential Scanning Calorimeter (DSC)

Differential scanning calorimetry measurements were used to determine the glass transition temperature (Tg) of wet and dry samples. Measurements were obtained using a TA Q200 Differential Scanning calorimeter (TA Instruments, New Castle, Del.). Hermetically sealed aluminum Tzero (Switzerland) pans with a hole poked in the top were used for all samples and goal foam sample weight was 5-10 mg. The cycle used for dry samples (Dry Tg) was first equilibrated at −40° C. for 5 min, heated to 120° C. (40° C./min) and cooled to −40° C. It was then reheated again at 10° C./min to 120° C. The second heating curve was analyzed using TA Universal Analysis software to determine dry Tg. Wet Tg foam samples were incubated in a 50° C. water bath for 30 minutes and pressed between Kimwipes to remove moisture prior to running. The cycle for wet samples was equilibrated at −40° C. for 5 minutes, with a single heating cycle that ramps to 100° C. at 10° C./min. The wet Tg was determined from the inflection point on the heating curve using TA Universal Analysis software.

4.5.2 Unconstrained Expansions

After cleaning and drying, 6 mm cylindrical biopsy punches of foams (n=3) were threaded over a wire and initial images were taken. The punches were radially crimped using Machine Solutions SC250 radial crimper heated to 100° C. The crimped foams were allowed to relax at room temperature in a desiccated container for 24 hours before studying their expansion behavior. Images of crimped foams were taken to determine initial crimped diameter. A water bath was heated to 37° C. and pictures were taken every minute until the 10 minute time point, then images were taken every five minutes until 30 minutes. All images were analyzed by measuring five points on each foam punch at every time point using ImageJ. Results were reported as percent expansion with standard deviation.

4.5.3 Tensile Testing

Rectangular foam samples were cut to approximately 25 mm×15 mm×3 mm and epoxied to wooden blocks at the short end. Epoxy was allowed to cure overnight and samples were stored under vacuum in a bell jar for at least 24 hours prior to tensile testing to ensure ambient moisture did not affect mechanical properties. A MTS Insight 30 Universal Tensile Tester (MTS Systems Corp, Eden Prairie, Minn.) with a 50 N load cell was used in tensile testing. Clamps were tightened on the wooden blocks at either end of the sample and machine was zeroed. The protocol used was a strain to failure at 5 mm/min constant strain rate.

4.6 Magnetic Resonance Imaging (MRI)

Pilot magnetic resonance imaging (MRI) validation studies of the SMP foams were performed at the Magnetic Resonance Systems Lab at Texas A&M University. Cylindrical foam samples (6 mm diameter) were placed in DI water in microcentrifuge tubes and a fish oil tablet was used as a positive control for images. High resolution multi-slice T₁-weighted images (TR 500 msec, TE 30 msec) were acquired on a 4.7T Varian Inova scanner using a standard spin-echo pulse sequence. Transverse images were taken with a 30 mm×60 mm field-of-view and 64×128 matrix size for 469 μm resolution in both x (left-right) and y (anterior-posterior) dimensions with a slice thickness of 1 mm. For coronal images, a 120 mm×80 mm field-of-view and 256×128 matrix size were used for 469 μm resolution in the z (head-foot) dimension and 625 μm resolution in the x dimension with a slice thickness of 1 mm. All other parameters were kept constant throughout all acquisitions. The raw data acquired from the scanner was imported to Matlab® for reconstruction and display of all images.

4.7 X-Ray Imaging

Foam samples were cut into blocks (˜10 mm×10 mm) of 10 mm and 5 mm thicknesses and cylinders of 6 mm diameter. All samples were arranged according to composition and imaged using OrthoScan C-arm system (Mobile DI Model 1000-0005). There were 6 mm diameter cylindrical prototype devices made as well in crimped and expanded forms. IMPEDE device (Shape Memory Medical, Santa Clara, Calif.) containing a Pt coil and marker band was used as a positive control. Samples were also imaged through a ½″ aluminum plate which served as a bone analog with respect to X-ray attenuation. Images were converted to 8-bit grayscale, processed to remove background, and analyzed using ImageJ to determine X-ray density. Sixty measurements were taken using the multi-point selector yielded values between 0 (black) and 255 (white). X-ray density was calculated from using the equation

$$X \text{-} Ray Density = \frac{255 - ( Image J Pixel Intensity Value )}{255} \qquad ( 2 )$$

such that a density of 1 would correlate to an entirely black image.

4.8 Mass Spectroscopy of Extractables

The extraction conditions chosen were simulated use, batch extraction with agitation. Cylindrical (6 mm diameter×25 mm length) SMP foam samples were incubated in 10 mL of extraction solution at 37° C. in an incubation shaker at 90 rpm. DI water was used as the polar extraction solution and a 50/50 water/ethanol solvent mixture was used as the non-polar extraction solution. After 72 hours, samples were removed and 1% nitric acid was added to solutions to ensure metallic particle stability before samples were analyzed using a Perkin Elmer NexION 300D inductively coupled mass spectroscopy (ICP-MS) instrument in the Elemental Analysis Laboratory at Texas A&M University.

4.9 Indirect Cytocompatibility

Using sterile biopsy punches in an aseptic environment, foams were cut into 8 cm diameter discs with 2 mm thickness. The surface area of the discs was calculated based on the pore size of each foam composition as previously performed by Weems et al. Foams were submerged in complete cell culture media for extraction at a ratio of 3 cm² foam per 1 mL of media for 72 hours at 37° C. in a rotating incubator. An additional tube with media only was included as a cytocompatible control. Complete cell culture media was made from Dulbecco's Modified Eagle Medium (DMEM, MilliporeSigma, St. Louis, Mo.) and supplemented with 10% newborn calf serum (NCS, MilliporeSigma, St. Louis, Mo.), 1% penicillin-streptomycin (P/S, VWR, Radnor, Pa.) solution, and 0.1% fungizone (VWR, Radnor, Pa.).

Balb/3T3 cells, clone A31 (3T3s, ATCC, Manassas, Va.) were thawed and passed at least once prior to plating for the cytocompatibility assay. All incubation periods were done in a humidified incubator at 37° C. with 5% CO2. Complete cell culture medium used for 3T3 culture and the assays was the same as described above for the extraction. 3T3s were harvested with trypsin and seeded in 96 well plates at 7,500 cells per well. After a 24-hour incubation, cell morphology and distribution were assessed, and images were acquired of each well using a Biotek Cytation5 Imaging Reader (Biotek, Winooski, Vt.). The media was then removed and replaced with media from the foam extractions or control. The cells were incubated with these treatments for 48 hours, then cell morphology and distribution were assessed and additional images were acquired of each well using the Cytation5. Finally, cell viability was measured indirectly using a resazurin assay to assess relative metabolic activity. Cells were incubated with 5% resazurin in complete media for 3 hours, then the fluorescence was measured using an excitation wavelength of 560 nm and an emission wavelength of 590 nm on a Tecan Infinite M200 Pro Plate Reader (Tecan, Morrisville, N.C.). Cell viability was calculated using the following equation:

$$Cell Viability ( X ) = \frac{RF {U}_{\frac{560}{590}} ( X ) - R F {U}_{\frac{560}{590}} ( blanks )}{R F {U}_{\frac{560}{590}} ( Untreated Control ) - RF {U}_{\frac{560}{590}} ( blanks )} \qquad ( 3 )$$

where X is any treatment group and RFU is relative fluorescent units (i.e., fluorescence intensity). Extractions and cytocompatibility assays were repeated in triplicate.

With the above discussion in mind, various embodiments are addressed below.

An embodiment includes the use of a SMP foam to seal or occlude an internal organ or skin biopsy site. The SMP foam may be deployed in a compact/crimped state to conform dynamically to fill the biopsy cavity to induce rapid thrombus formation acutely, and chronically serve as a biocompatible scaffold for long-term tissue regeneration. Such an embodiment may be coupled with a hydrogel and/or gelatin plug.

The SMP foam may include polyurethane but other embodiments may include polyurea, polyamide, polyester, polycaprolactone, or combinations thereof.

An embodiment is biodegradable or biodurable.

The shape memory nature of the material in an embodiment allows the material to remain in a compact secondary state within a delivery system and to facilitate easy, minimally invasive deployment into the biopsy site. The primary shape of the material will allow the device to expand and conform to the biopsy site to maximize healing and the formation of a biologic seal. Oversizing the device to the biopsy site would enable a friction fit within the biopsy site and local compression for faster embolization. The biocompatibility of the material and open celled structure of the foam matrix will maximize incorporation of the scaffold into the native tissue.

An SMP foam cylinder could be either preloaded into the same syringe/delivery system as the biopsy needle or inserted directly through the syringe/biopsy needle after the biopsy is taken such that the foam is delivered immediately following the biopsy. Upon exiting the needle at the biopsy site, the foam immediately expands to completely fill the tract left behind by the biopsy.

In an embodiment the SMP foam is a reaction product of N,N,N′,N′-Tetrakis (2-hydroxypropyl) ethylenediamine (HPED); 2,2′,2″-nitrilotriethanol (TEA); and 1,6-diisocyanatohexane (HDI).

An embodiment may be used to fill tracts left behind after organ biopsies. It could also be used as a hemostatic agent to prevent excessive bleeding after trauma that caused hemorrhage. An embodiment may include a SMP-hydrogel composite device to allow the incorporation of antibacterial properties into the device or delivery of pro-healing compounds. Alternative embodiments could include varying density foams to improve sealing or prevent expulsion of the plug from the biopsy site.

In one embodiment, the biopsy needle is used to core the sample from the organ, and the specimen is removed from the needle via suction or mechanical means while the biopsy needle stays in position. The SMP plug is positioned in the distal portion of the biopsy needle with a pusher and held in place while the biopsy needle is retracted to unsheathe the SMP plug material. Once implanted in the body, the plug radially expands to occlude and seal the biopsy tract. The foam can be introduced through a multilumen adapter that is used to remove the biopsy sample and deliver the SMP plug. Alternatively, the biopsy sample can be removed, and a single lumen foam adapter can be placed on the biopsy needle to introduce the foam into the biopsy needle lumen. In an embodiment the SMP plug is sized to be approximately the same length as the removed biopsy sample. In general, the expanded SMP foam plug will be oversized to the biopsy tract.

In an embodiment the system includes a secondary conduit that is introduced co-axially to the biopsy needle to introduce the SMP plug into the biopsy tract.

In an embodiment after biopsy sample removal, a flexible rod is placed within the lumen of the biopsy needle and sits within the biopsy tract as the needle is retracted. The flexible rod is used to guide a delivery sheath back into the same conduit left by the biopsy needle to introduce the SMP plug.

In an embodiment the delivery conduit (biopsy needle or delivery sheath) is placed at the surface of the organ at the origin of the biopsy tract, and the SMP foam plug is pushed into the biopsy tract.

An embodiment includes a marked delivery pusher system that helps the physician introduce the SMP plug at the appropriate depth before biopsy needle or delivery sheath retraction. Alternatively, it can be used to push the SMP foam plug out of the delivery conduit the appropriate distance.

Embodiments provide a biopsy sealant device and technique that offer true visibility under X-Ray or MRI. This enables one to precisely locate the treatment devices and the tissue of interest during follow-up imaging. The biopsy sealant device and technique reduce complications by both sealing effectively and offering imaging contrast so that, for example, tumor changes can be measured relative to the biopsy location. Postbiopsy the preferred imaging method for monitoring tumor/mass progress is MRI but X-ray may be preferred during biopsy sealing.

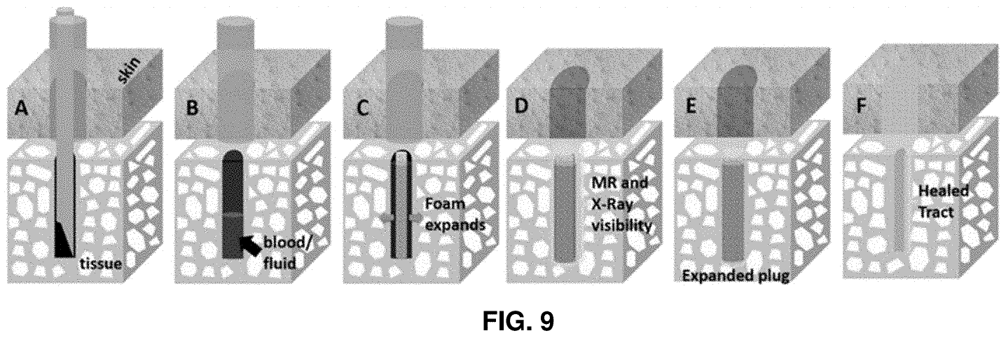

FIG. 9 shows how an embodiment is implemented for biopsy tract sealing. The organ tissue is collected via a percutaneous biopsy, wherein the cannula is placed over the site and the biopsy needle is inserted at the desired site (Stage A). The cannula is left in place and blood/fluid may fill the biopsy tract depending on tissue type (Stage B). An SMP foam plug is deployed through the cannula into the biopsy tract in a crimped state (Stage C). The mixture of blood/fluid at physiological temperature drives the expansion of the foam-gel plug. Additionally, the SMP foam is visible under radiographic and MRI modalities, where the X-ray and MRI contrast are in the foam (Stage D). As the sealed tract progresses, the SMP triggers the wound healing cascade at the tract, leading to a natural closure of the tract site. The foam-gel plug will degrade at the site proportional to the advent of new tissue (Stage E).

Such an embodiment has advantages over conventional sealants.

For example, conventional sealants include saline, autologous blood patch, collagen or hydrogel plugs, and fibrin glue. In particularly difficult cases or in patients with abnormal blood coagulation, embolic coils may be used in liver biopsies resulting in severe hemorrhage. There is also off-label use of Gelfoam or Surgifoam reported in the literature. However, none of these methods offer any advantage with imaging or the reduction of pneumothoraces. With the advent of improved early detection modalities and the increasing number of biopsies, it is crucial that biopsy sealants align to this change by offering visibility under multiple modalities. The dual-modality imaging capabilities of embodiments described herein is a major benefit compared to existing technologies. These materials will offer visibility with both X-ray and MR imaging modalities that will permit tracking of the biopsy site for six-to-twelve months.

Applicant realized several obstacles existed and must be overcome before SMP foam embodiments that include both ATIPA and GPA could be realized. Applicant addressed these obstacles as follows.

For example, the use of ATIPA with foams based on HT, BEP, MPD, and HDI raises solubility issues. Further solubility issues were raised when GPA was included in foams that were based on ATIPA, HT, BEP, MPD, and HDI. However, embodiments addressed the solubility issues through, for example, various process parameters such as longer mixing times, longer time on shaker plates, and not adding ATIPA and GPA at the same time. More specifically, in an embodiment the GPA is added to the OH premix just before the NCO and OH premixes are combined for foaming.

Further, Applicant also realized issues exist with adding gadolinium to foams that were based on ATIPA, HT, BEP, MPD because carboxylic acids on the GPA cause production of carbon dioxide when combined with isocyanates. Applicant addressed this by, in an embodiment, adding in the GPA just before foaming (otherwise premature foaming could occur).

Applicant further worked to ensuring the combination of material density and amount of imaging monomers (ATIPA or GPA) added were sufficient or in the correct range for MRI. Using T₁ weighted parameters it is possible to have too much GPA. For example, a 20 AT 20 GPA composition did not have the desired brightening effect because of the high amount of GPA. As noted above, Applicant determined gadolinium causes shortening of both T₁ and T₂. When using T₁-weighted imaging sequences, the T₁ shortening manifests as the clinically desired image brightening, while T₂ shortening will result in image darkening. Depending on the concentration of gadolinium used, one of these effects will be dominant. The 20 AT 20 GPA composition had too high of a concentration of gadolinium and T₂ effects dominated, so it was excluded from further study.

Conversely, for X-ray imaging Applicant determined a high concentration of ATIPA (or other radiopaque monomer) and high material density are desired for imaging. Further, density may have a dominant role in X-ray visibility as compared to other factors.

The following examples pertain to further embodiments.

Example 1. A system comprising: a thermoset shape memory polymer (SMP) foam; wherein the SMP foam is chemically bonded to both: (a) iodine, and (b) a gadolinium-based contrast agent (GBCA); wherein: (a) the SMP foam is configured to expand from a compressed secondary state to an expanded primary state in response to thermal stimulus, and (b) the SMP foam is a poly(urethane-urea-amide).

In some embodiments an imaging element may be physically included with the foam. For example, a nanoparticle or microparticle filler may be included with the foam. However, such a form of inclusion may make the element susceptible to migration away from the biopsy tract. In contrast, chemical bonding of the element (e.g., covalent bonding) to the foam may be more secure and thereby prevent the element (e.g., iodine) from migrating away from the foam. Also, some embodiments may achieve physical inclusion by incorporating the element into a layer that covers some of the foam, but such a layer may inhibit foam expansion. Embodiments addressed herein include elements that chemically bond to the foam (e.g., within the foam's polymer backbone or in a sidechain of the foam polymer) but do not overly diminish foam mechanical properties, such as foam expansion rate or whether the foam fully expands.

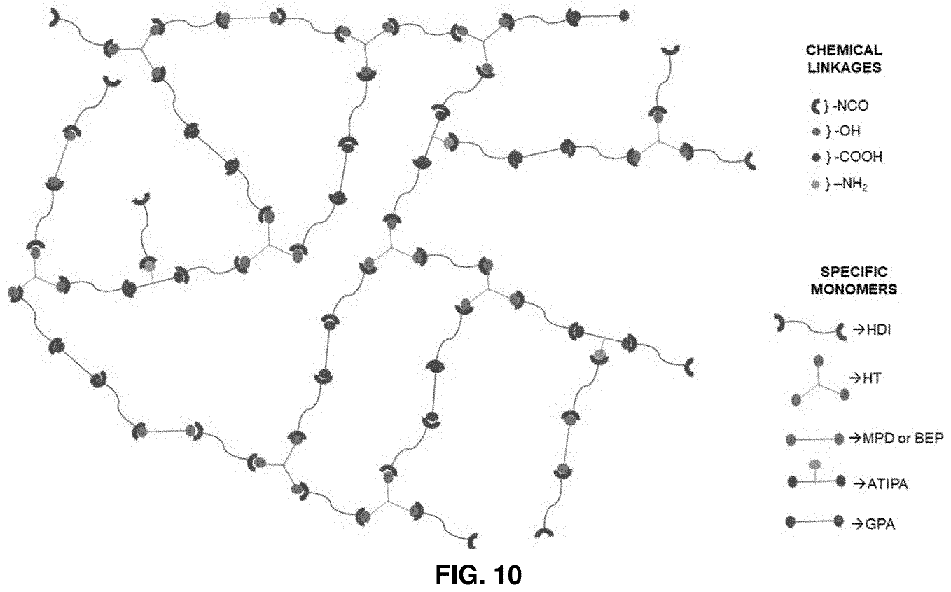

Regarding chemical bonding, see FIG. 10 for an embodiment addressing chemical bonding to provide an X-ray and MRI visible foam.

When compared to particulate additives, the chemical approach of incorporating contrast monomers (e.g., triiodobenzene monomers) into the foam during synthesis enables higher contrast loading percentages without overly affecting the mechanical integrity or characteristics (e.g., particulate additives serve as stress concentrations and reduce tensile strength of an excessively loaded composite) of the bulk material. This SMP material system can be utilized to create low density foams for applications without the need for metal components such as platinum backbones or marker bands. Embodiments of this material system allow for entirely polymeric, biodurable devices used for a variety of applications, including biopsy plugs. Incorporating aromatic diisocyanates also creates a very rigid polymer system appropriate for bone tissue applications that require x-ray visibility. Degradable linkages such as ethers, esters, or tertiary amines are incorporated in some embodiments to create a biodegradable material formulation.

The x-ray contrast of the ATIPA molecule is derived from the triiodobenzene motif, which incorporates three high-z iodine atoms. It is terminated with a primary aromatic amine and two carboxylic acids, giving it a functionality of three for crosslinking reactions with isocyanates. Further, the reaction between isocyanates and carboxylic acids yields an amide linkage and carbon dioxide, making ATIPA a chemical blowing agent during foam polymerization.

An alternative version of Example 1. A system comprising: a thermoset shape memory polymer (SMP) foam; wherein the SMP foam is chemically bonded to: (a) iodine, and (b) a magnetic resonance (MR) contrast agent; wherein: (a) the SMP foam is configured to expand from a compressed secondary state to an expanded primary state in response to thermal stimulus, and (b) the SMP foam is a poly(urethane-urea-amide).

MR contrast agents may include gadolinium, iron (e.g., superparamagnetic iron-platinum particles (SIPPs)), manganese (e.g., manganese chelates such as Mn-DPDP), bromine (e.g., perfluorooctyl bromide), or combinations thereof. Some of these agents may be chemically bonded to the foam while others are physically included with the foam.

Another version of Example 1. A system comprising: a thermoset shape memory polymer (SMP) foam; wherein the SMP foam is chemically bonded to both: (a) iodine, and (b) a gadolinium-based contrast agent (GBCA); wherein the SMP foam is configured to expand from a compressed secondary state to an expanded primary state in response to thermal stimulus.

An alternative version of Example 1. A system comprising: a thermoset shape memory polymer (SMP) foam; wherein the SMP foam is chemically bonded to: (a) iodine, and (b) a magnetic resonance (MR) contrast agent; wherein the SMP foam is configured to expand from a compressed secondary state to an expanded primary state in response to thermal stimulus.

Example 2. The system of Example 1 wherein the SMP foam is radiopaque.

Example 3 is skipped.

Example 4. The system of Example 1 wherein the SMP foam has magnetic resonance imaging (MRI) visibility.

Example 5. The system of Example 4 wherein the SMP foam is more MRI visible than deionized water and less MRI visible than fish oil.

Example 6. The system of Example 1 wherein the SMP foam has X-ray visibility and magnetic resonance imaging (MRI) visibility.

Whether something is “x-ray visible” or “radiopaque” or “MR visible” or “MRI visible” is judged according to a person of ordinary skill in the art, such as a physician that routinely takes biopsies and/or follows up on biopsy-related tissue using imaging, such as fluoroscopy or MRI. While imaging power may vary depending on the imaging machine used and the like, a person of ordinary skill in the art will still understand whether a foam is visible under normal clinical conditions such that the foam is discernable from the surrounding anatomy.

Example 6.1 The system of Example 1 wherein the SMP foam has, simultaneously, X-ray visibility and magnetic resonance imaging (MRI) visibility.

For example, SMP foam may be resident in a delivery tube or conduit while in storage. At that time, before any plasticization or transformation of the foam occurs, the foam would be both X-Ray visible and MRI visible. The same is true after the foam is implanted in a biopsy track and has expanded fully or partially (i.e., the foam would be both X-Ray visible and MRI visible when implanted and expanded partially or fully).

Example 6.2 The system of Example 1 wherein the SMP foam has, simultaneously, X-ray visibility and magnetic resonance imaging (MRI) visibility when the SMP foam is in the compressed secondary state.

Example 6.3 The system of Example 6.2 wherein the SMP foam has, simultaneously, X-ray visibility and MRI visibility when the SMP foam is in the expanded primary state.

In an embodiment Xray opacity diminishes during expansion. Applications for such a feature include, without limitation, endovascular applications. For instance, a user may visualize the compressed device during delivery to an aneurysm. However, after deployment the expanded device is radiolucent (or at least more radiolucent than the compressed device) and allows the user easy angiography visualization within the aneurysm after treatment.

Example 7. The system according to any of Examples 1-6.3 wherein the iodine is included in a triiodobenzene monomer.

Example 8. The system of Example 7 wherein the triiodobenzene monomer includes at least one of (a) 5-amino-2,4,6-triiodoisophthalic acid (ATIPA), (b) diatrizoic acid, (c) iohexol, (d) triiodophenol, or (e) combinations thereof.

Example 9. The system of Example 8 wherein the triiodobenzene monomer includes ATIPA.

Another version of Example 9. The system of Example 8 wherein the triiodobenzene monomer includes diatrizoic acid.

Another version of Example 9. The system of Example 8 wherein the triiodobenzene monomer includes iohexol.

Another version of Example 9. The system of Example 8 wherein the triiodobenzene monomer includes triiodophenol.

Example 10. The system of Example 9 wherein the triiodobenzene monomer includes ATIPA and the ATIPA crosslinks polymer chains of the SMP foam.

Example 11. The system according to any of Examples 1-10 wherein: the SMP foam includes at least one of platinum, tungsten, tantalum, or combinations thereof; the at least one of platinum, tungsten, tantalum, or combinations thereof being physically bound within the SMP foam.

Example 12. The system of Example 11 wherein the at least one of platinum, tungsten, tantalum, or combinations thereof is not chemically bound to the SMP foam.

Example 13. The system according to any of Examples 1-12 comprising a backbone that traverses the SMP foam, wherein the backbone includes at least one of a polymer filament, a metal, or combinations thereof.

Example 14. The system of Example 13 wherein the backbone includes the polymer filament and no metal.

Another version of Example 14. The system of Example 13 wherein the backbone includes the metal.

Another version of Example 14. The system of Example 13 wherein the system includes no metal.

For instance, the system may be the deployable and implantable portion of a larger system. However, the portion that is finally implanted in the patient (i.e., the system) includes no metal. This may be due to the X-Ray and/or MRI visibility aspects of the system.

Example 15. The system according to any of Examples 1-10 wherein the GBCA includes gadopentetic acid (GPA).

In an embodiment, MRI visibility diminishes with time. For example, this visibility diminishment may occur due to a breakdown in gadolinium chelation over time in-vivo. More generally, gadolinium may diffuse away from the foam over time. As a result, MRI visibility dropoff may be evidence of or a function of biodegradation.

Example 16. The system according to Example 15 wherein the SMP foam includes at least one of platinum, tungsten, tantalum, or combinations thereof, the at least one of platinum, tungsten, tantalum, or combinations thereof being physically bound within the SMP foam.

Example 17. The system of Example 16 wherein the at least one of platinum, tungsten, tantalum, or combinations thereof is not chemically bound to the SMP foam.

Example 18. The system according to any of Examples 11-14 comprising a backbone that traverses the SMP foam, wherein the backbone includes at least one of a polymer filament, a metal, or combinations thereof.

Example 19. The system of Example 18 wherein the backbone includes a polymer filament and no metal.

Example 20. The system according to any of Examples 1-19 comprising a conduit, wherein the SMP foam is included within the conduit.

Another version of Example 20. The system according to any of Examples 1-19 comprising a biopsy seal system, wherein: the biopsy seal system includes a conduit; the SMP foam is included within the conduit; the SMP foam is configured to seal a biopsy tract.

Another version of Example 20. The system according to any of Examples 1-19 comprising an anatomic void seal system, wherein: the anatomic void seal system includes a conduit; the SMP foam is included within the conduit; the SMP foam is configured to seal the anatomic void.

Example 21. The system of Example 20 comprising a rod, wherein: the conduit includes a minimum internal diameter; the rod includes a maximum outer diameter; the maximum outer diameter is less than the minimum inner diameter, and the rod is configured to slide within the conduit to push the SMP foam out of the conduit.

Example 22. The system according to any of Examples 20-21 comprising: an additional thermoset SMP foam; wherein the additional SMP foam is chemically bonded to both: (a) iodine, and (b) a GBCA; wherein: (a) the additional SMP foam is configured to expand from a compressed secondary state to an expanded primary state in response to thermal stimulus, and (b) the additional SMP foam is a poly(urethane-urea-amide).

Example 23. The system according to Example 22 wherein the conduit includes the additional SMP foam.

Example 24. The system according to any of Examples 1 to 23 wherein the SMP foam is a reaction product of an aliphatic monomer and a diisocyanate.

Example 25. The system of Example 24 wherein the aliphatic monomer comprises at least one of (a)(i) multiple amine functional groups, (a)(ii) multiple alcohol functional groups, (a)(iii) multiple carboxylic acid functional groups, or (a)(iv) combinations thereof.

Example 26. The system of Example 25 wherein the aliphatic monomer includes at least one of 1,2,6-hexanetriol (HT); 2-butyl-2-ethyl-propanediol (BEP); 3-methyl-1,5-pentanediol (MPD); diethylene glycol (DEG); triethylene glycol (TEG); triethanolamine (TEA); tetrakis-hydroxypropyl ethylenediamine (HPED); glycerol; trimethylolpropane; trimethylolmethane; 1,2,4-butanetriol; 1,2-diaminopropane; 2,2-Dimethyl-1,3-propanediamine; 1,8-Diaminooctane; 3-Amino-1,2-propanediol; 2-Amino-2-methyl-1,3-propanediol; 1,3-Diamino-2-propanol; aspartic acid; or combinations thereof.

Another version of Example 26. The system of Example 25 wherein the aliphatic monomer includes 1,2,6-hexanetriol (HT); 2-butyl-2-ethyl-propanediol (BEP); and 3-methyl-1,5-pentanediol (MPD).

Another version of Example 26. The system of Example 25 wherein the aliphatic monomer includes glycerol.

Another version of Example 26. The system of Example 25 wherein the aliphatic monomer includes triethanolamine (TEA) and tetrakis-hydroxypropyl ethylenediamine (HPED).

Another version of Example 26. The system of Example 25 wherein the aliphatic monomer includes triethanolamine (TEA).

Another version of Example 26. The system of Example 25 wherein the aliphatic monomer includes tetrakis-hydroxypropyl ethylenediamine (HPED).

Example 27. The system of Example 26 wherein the diisocyanate includes at least one of hexamethylene diisocyanate (HDI); trimethylhexamethylene diisocyanate (TMHDI); isophorone diisocyanate (IPDI); 1,3,4-triisocyanato-2,4,6-trimethylbenzene; toluene diisocyanate; methylene diphenyl diisocyanate; or combinations thereof.

Another version of Example 27. The system of Example 26 wherein the diisocyanate includes hexamethylene diisocyanate (HDI).

Another version of Example 27. The system of Example 26 wherein the diisocyanate includes trimethylhexamethylene diisocyanate (TMHDI).

Another version of Example 27. The system of Example 26 wherein the diisocyanate includes hexamethylene diisocyanate (HDI) and trimethylhexamethylene diisocyanate (TMHDI).

Example 28. The system according to any of Examples 1-27 wherein the SMP foam has a dry Tg above 37 degrees Celsius and a wet Tg below 37 degrees Celsius.

Dry Tg may be determined using a TA 0200® Differential Scanning calorimeter on 5-10 mg foam samples in a vented aluminum pan. The samples may be equilibrated at −40° C. for 5 minutes, then heated to 120° C., cooled to −40° C., and reheated to 120° C. at temperature ramps of 10° C./min. Tg may be calculated at the inflection point of the second heating curve.

Example 28 recites a dry Tg that is calculated using this process described in the paragraph immediately above (i.e., dry Tg as recited in the Examples is to be calculated using the above test regarding time, temperature, process, and inflection point of the second heating curve).

Wet Tg foam samples may be immersed in 50° C. water for 30 minutes to achieve moisture plasticization. Moisture may be removed by compressing the foam between tissue paper at 2 tons for 30 seconds using a Carver® laboratory press. 5-10 mg foam samples may be added to an aluminum pan and hermetically sealed. Samples may then be cooled to −40° C., equilibrated for 5 minutes, and heated to 100° C. at 10° C./min. Wet Tg may then be calculated from the heating curve inflection point.

In some embodiments the dry Tg is between 40 and 100 or 40 and 90 or 40 and 70 or 40 and 60 C. In some embodiments the SMP foam has a moisture plasticized glass transition temperature onset below 37 C but in other embodiments the moisture plasticized glass transition temperature onset is below 40, 39, 38, 37, 36, 35, 34 C.

Example 25. A system comprising: a biopsy seal system; wherein the biopsy seal system includes: (a) a radially-compressed, porous, open-cell, partially reticulated, thermoset, shape memory polymer (SMP) foam, and (b) a conduit that includes the SMP foam; wherein the SMP foam is chemically bonded to bot: (a) iodine, and (b) a gadolinium-based contrast agent (GBCA); wherein: (a) the SMP foam is configured to expand from a compressed secondary state to an expanded primary state in response to thermal stimulus, and (b) the SMP foam is a poly(urethane-urea-amide).

For “partially reticulated”, the foam may include first and second cells that share and directly contact a ring of struts that provide structural support for the first and second cells. A membrane directly contacts the ring of struts, and the membrane is partially reticulated but not fully reticulated. The partially reticulated membrane includes: (a)(i) a void that forms a path configured to allow fluid to flow between the first and second cells, (a)(ii) an interface, between the partially reticulated membrane and the void, which is uneven. The ring of struts defines an outer perimeter of the membrane and the void defines an inner perimeter of the membrane. An outer membrane area for the membrane is an area bounded by the outer perimeter defining an area of the membrane before reticulation. A void area is an area bounded by the inner perimeter defining an area of the void. The void area is between 25% and 75% of the outer membrane area. In other words, the membrane is “partially reticulated”.

A “conduit” need not be a pipe or something so restricted and may include, for example, a rod or substrate with a channel or groove with the foam within the channel or groove.

Foam morphology, such as pore size and density, is tailored via changes in foam premix viscosity and by altering the amount of physical blowing agent, surfactants, and catalysts during synthesis. The ability to independently control these material properties increases the utility of x-ray and MR visible embolic foams by opening avenues for device optimization to meet specific application needs.

Example 1a. A method comprising: providing a triiodobenzene monomer; providing a gadolinium-based contrast agent (GBCA); providing an aliphatic monomer comprising at least one of: (a)(i) multiple amine functional groups, (a)(ii) multiple alcohol functional groups, (a)(iii) multiple carboxylic acid functional groups, or (a)(iv) combinations thereof; providing a diisocyanate; mixing the triiodobenzene monomer, the GBCA, the aliphatic monomer, and the diisocyanate into a solution; forming a thermoset shape memory polymer (SMP) foam from the solution.

Example 2a. The method of Example 1a wherein: the triiodobenzene monomer includes at least one first member selected from the group consisting of 5-amino-2,4,6-triiodoisophthalic acid (ATIPA), diatrizoic acid, iohexol, triiodophenol, or combinations thereof; the aliphatic monomer includes at least one second member selected from the group consisting of 1,2,6-hexanetriol (HT); 2-butyl-2-ethyl-propanediol (BEP); 3-methyl-1,5-pentanediol (MPD); diethylene glycol (DEG); triethylene glycol (TEG); triethanolamine (TEA); tetrakis-hydroxypropyl ethylenediamine (HPED); glycerol; trimethylolpropane; trimethylolmethane; 1,2,4-butanetriol; 1,2-diaminopropane; 2,2-Dimethyl-1,3-propanediamine; 1,8-Diaminooctane; 3-Amino-1,2-propanediol; 2-Amino-2-methyl-1,3-propanediol; 1,3-Diamino-2-propanol; aspartic acid, or combinations thereof; the diisocyanate includes at least one third member selected form the group consisting of hexamethylene diisocyanate (HDI); trimethylhexamethylene diisocyanate (TMHDI); isophorone diisocyanate; 1,3,4-triisocyanato-2,4,6-trimethylbenzene; toluene diisocyanate; methylene diphenyl diisocyanate, or combinations thereof.

Example 3a. The method of Example 2a wherein the at least one second member is selected from the group consisting of HT; BEP; MPD; DEG; TEG; TEA; HPED; glycerol; trimethylolpropane; trimethylolmethane; 1,2,4-butanetriol, or combinations thereof.

Example 4a. The method of Example 2a wherein the at least one second member is selected from the group consisting of 1,2-diaminopropane; 2,2-Dimethyl-1,3-propanediamine; 1,8-Diaminooctane; 3-Amino-1,2-propanediol; 2-Amino-2-methyl-1,3-propanediol, or combinations thereof.

Example 5a. The method according to any of Examples 2a-4a wherein the at least one third member is selected form the group consisting of HDI; TMHDI; isophorone diisocyanate, or combinations thereof.

Example 6a. The method according to any of Examples 2a-4a wherein the at least one third member is selected form the group consisting of elected form the group consisting of 1,3,4-triisocyanato-2,4,6-trimethylbenzene; toluene diisocyanate; methylene diphenyl diisocyanate; combinations thereof.

Example 7a. The method according to any of Examples 2a-6a wherein the at least one first member includes ATIPA.

Example 8a. The method according to any of Example 2a-7a, wherein the GBCA includes gadopentetic acid (GPA).

Example 9a. The method according to any of Example 2a-7a comprising crosslinking the at least one second and third members with the at least one first member.

Example 10a. The method according to any of Examples 2a-9a wherein forming the SMP foam from the solution comprises utilizing the at least one first member as a chemical blowing agent.

Example 11a. The method according to any of Examples 2a-10a wherein the aliphatic monomer includes at least one fourth member selected from the group consisting of HT; BEP; MPD; DEG; TEG; TEA; HPED; glycerol; trimethylolpropane; trimethylolmethane; 1,2,4-butanetriol; 1,2-diaminopropane; 2,2-Dimethyl-1,3-propanediamine; 1,8-Diaminooctane; 3-Amino-1,2-propanediol; 2-Amino-2-methyl-1,3-propanediol; 1,3-Diamino-2-propanol; aspartic acid; or combinations thereof.

Example 1b. A system comprising: an iodine and gadolinium containing thermoset open-cell shape memory polymer (SMP) foam that is both x-ray visible and magnetic resonance (MR) visible; wherein (a) the SMP foam is configured to expand from a compressed secondary state to an expanded primary state in response to thermal stimulus, (b) the SMP foam is a poly(urethane-urea-amide).

Example 2b. The system of Example 1 b wherein the iodine is included in a triiodobenzene monomer and the iodine and gadolinium are both covalently bonded within a polymer network of the SMP foam.

Example 3b. The system according to any of Examples 1-2b wherein the SMP foam in the secondary state contains between 50 and 500 mg/ml of Iodine.

Example 4b. The system according to any of Examples 1-3b wherein: the SMP foam in its primary state has a density of less than 0.1 g/cc; the SMP foam has a dry glass transition temperature (Tg) between 30 and 100 degrees C.

Example 5b. The system according to any of Examples 1-4b wherein the SMP foam comprises polycaprolactone (PCL).

Example 1c. A method comprising: performing a biopsy to remove cells from a tissue; forming a void in the tissue in response to performing the biopsy; locating a first conduit within the void; deploying a shape memory polymer (SMP) foam from the first conduit into the void to at least partially seal the void; using X-ray to image the SMP foam; using magnetic resonance (MR) to image the SMP foam; wherein the SMP foam is a porous, open-cell, partially reticulated, thermoset foam; wherein the SMP foam is chemically bonded to both: (a) iodine, and (b) a gadolinium-based contrast agent (GBCA); wherein: (a) the SMP foam is configured to expand from a compressed secondary state to an expanded primary state in response to thermal stimulus, and (b) the SMP foam is a poly(urethane-urea-amide).

Example 2c. The method of Example 1c comprising: locating a second conduit within the void; inserting the first conduit within the second conduit before deploying the SMP foam into the void.

Example 3c. The method of Example 2c comprising; inserting the second conduit into the tissue before performing the biopsy; locating the cells within the second conduit; separating the cells from the tissue while the cells are located within the conduit.

Example 4c. The method of Example 3c comprising using X-ray to image the SMP foam more than 1 week before using MR to image the SMP foam.

Example 5c. The method of Example 4c comprising using the SMP foam as a fiducial marker when using MR to image the SMP foam.

The foregoing description of the embodiments of the invention has been presented for the purposes of illustration and description. It is not intended to be exhaustive or to limit the invention to the precise forms disclosed. This description and the claims following may include terms, such as left, right, top, bottom, over, under, upper, lower, first, second, etc. that are used for descriptive purposes only and are not to be construed as limiting. For example, terms designating relative vertical position refer to a situation where a side of a substrate is the “top” surface of that substrate; the substrate may actually be in any orientation so that a “top” side of a substrate may be lower than the “bottom” side in a standard terrestrial frame of reference and still fall within the meaning of the term “top.” The term “on” as used herein (including in the claims) does not indicate that a first layer “on” a second layer is directly on and in immediate contact with the second layer unless such is specifically stated; there may be a third layer or other structure between the first layer and the second layer on the first layer. The embodiments of a device or article described herein can be manufactured, used, or shipped in a number of positions and orientations. Persons skilled in the relevant art can appreciate that many modifications and variations are possible in light of the above teaching. Persons skilled in the art will recognize various equivalent combinations and substitutions for various components shown in the Figures. It is therefore intended that the scope of the invention be limited not by this detailed description, but rather by the claims appended hereto.

What is claimed is:

1. A system comprising:
a thermoset shape memory polymer (SMP) foam;
wherein the SMP foam is chemically bonded to both: (a) iodine, and (b) a gadolinium-based contrast agent (GBCA);
wherein: (a) the SMP foam is configured to expand from a compressed secondary state to an expanded primary state in response to thermal stimulus, and (b) the SMP foam is a poly(urethane-urea-amide).

2. The system of claim 1 wherein the SMP foam is radiopaque.

3. The system of claim 2 wherein the SMP foam is more MRI visible than deionized water and less MRI visible than fish oil.

4. The system of claim 1 wherein the SMP foam has, simultaneously, X-ray visibility and magnetic resonance imaging (MRI) visibility when the SMP foam is in the compressed secondary state.

5. The system of claim 4 wherein the SMP foam has, simultaneously, X-ray visibility and MRI visibility when the SMP foam is in the expanded primary state.

6. The system of claim 1 wherein the iodine is included in a triiodobenzene monomer.

7. The system of claim 6 wherein the triiodobenzene monomer includes at least one of (a) 5-amino-2,4,6-triiodoisophthalic acid (ATIPA), (b) diatrizoic acid, (c) iohexol, (d) triiodophenol, or (e) combinations thereof.

8. The system of claim 7 wherein the GBCA includes gadopentetic acid (GPA).

9. The system of claim 1 comprising a backbone that traverses the SMP foam, wherein the backbone includes a polymer filament.

10. The system of claim 1 wherein the GBCA includes gadopentetic acid (GPA).

11. The system of claim 1 comprising a biopsy seal system, wherein:
the biopsy seal system includes a conduit;
the SMP foam is included within the conduit; and
the SMP foam is configured to seal a biopsy tract.

12. The system of claim 11 comprising a rod, wherein:
the conduit includes a minimum internal diameter;
the rod includes a maximum outer diameter;
the maximum outer diameter is less than the minimum inner diameter; and
the rod is configured to slide within the conduit to push the SMP foam out of the conduit.

13. The system of claim 11 comprising:
an additional thermoset SMP foam;
wherein the additional SMP foam is chemically bonded to both: (a) iodine, and (b) a GBCA; and
wherein: (a) the additional SMP foam is configured to expand from a compressed secondary state to an expanded primary state in response to thermal stimulus, and (b) the additional SMP foam is a poly(urethane-urea-amide).

14. The system of claim 13 wherein the conduit includes the additional SMP foam.

15. The system of claim 1 wherein:
the SMP foam is a reaction product of an aliphatic monomer and a diisocyanate; and
the aliphatic monomer includes at least one of 1,2,6-hexanetriol (HT); 2-butyl-2-ethyl-propanediol (BEP); 3-methyl-1,5-pentanediol (MPD); diethylene glycol (DEG); triethylene glycol (TEG); triethanolamine (TEA); tetrakis-hydroxypropyl ethylenediamine (HPED); glycerol; trimethylolpropane; trimethylolmethane; 1,2,4-butanetriol; 1,2-diaminopropane; 2,2-Dimethyl-1,3-propanediamine; 1,8-Diaminooctane; 3-Amino-1,2-propanediol; 2-Amino-2-methyl-1,3-propanediol; 1,3-Diamino-2-propanol; aspartic acid; or combinations thereof;
wherein the diisocyanate includes at least one of hexamethylene diisocyanate (HDI); trimethylhexamethylene diisocyanate (TMHDI); isophorone diisocyanate (IPDI); 1,3,4-triisocyanato-2,4,6-trimethylbenzene; toluene diisocyanate; methylene diphenyl diisocyanate; or combinations thereof.

16. The system of claim 15 wherein the SMP foam has a dry T_{g} above 37 degrees Celsius and a wet T_{g} below 37 degrees Celsius.

17. A system comprising:
a biopsy seal system;
wherein the biopsy seal system includes: (a) a radially-compressed, porous, open-cell, partially reticulated, thermoset, shape memory polymer (SMP) foam, and (b) a conduit that includes the SMP foam;
wherein the SMP foam is chemically bonded to both: (a) iodine, and (b) a gadolinium-based contrast agent (GBCA); and
wherein: (a) the SMP foam is configured to expand from a compressed secondary state to an expanded primary state in response to thermal stimulus, and (b) the SMP foam is a poly(urethane-urea-amide).

18. The system of claim 17 wherein the iodine is included in a triiodobenzene monomer and the iodine and gadolinium are both covalently bonded within a polymer network of the SMP foam.

19. The system of claim 18 wherein the SMP foam in the secondary state contains between 50 and 500 mg/ml of Iodine.

20. The system of claim 19 wherein:
the SMP foam in its primary state has a density of less than 0.1 g/cc; and
the SMP foam has a dry glass transition temperature (T_{g}) between 30 and 100 degrees C.

21. The system of claim 19 wherein the SMP foam comprises polycaprolactone (PCL).
